(19) United States
(12) Patent Application Publication
(10) Pub. No.: US 2007/0099889 A1
(43) Pub. Date: May 3, 2007

(54) Use of pseudan and pseudan inclusion bodies

(76) Inventor: Paulette Royt, Fairfax, VA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/483,177

(22) Filed: Jul. 10, 2006

Related U.S. Application Data
(60) Provisional application No. 60/697,388, filed on Jul. 8, 2005.

(51) Int. Cl. A61K 31/555

(52) U.S. Cl. 514/187

(57) ABSTRACT

The present invention provides compositions and methods for reducing and/or inhibiting biofilm formation using a pseudan inclusion body.

This application claims the benefit of U.S. Provisional Application Ser. No. 60/697,388, filed Jul. 8, 2005, the entire disclosure of which is hereby incorporated by reference.

### SUMMARY OF THE INVENTION

A biofilm is a population or a community of microorganisms adhering to environmental surfaces in the form of multicellular aggregates. Although biofilms can be made up of a single species of microorganisms, in the natural setting, they often involve a mixture of species. Biofilms can form in almost any hydrated environment that has the proper nutrient conditions, and can develop on a wide variety of abiotic hydrophobic and hydrophilic surfaces, including glass, metals, and plastics. Biofilms also readily form on biotic surfaces including human skin and epithelial cells. Generally, surface material does not strongly affect biofilm growth. Examples of bacterial biofilms are chronic P. aeruginosa infections in the lungs of cystic fibrosis patients, oral microbes on teeth, the “slime” layer on the surface of submerged objects in aquatic environments, biofouling of water supply, sewage, and oil pipelines, and bacterial colonization of plant surfaces. A detailed review of biofilm structure and architecture is provided by Dunne et al. (Clinical Microbiology Reviews, vol. 15, pages 155-166, 2002).

It is now appreciated that the formation and maintenance of structured multicellular communities critically depends upon the production of extracellular substances that, in conglomerate, constitute an extracellular matrix. Genetic studies using biofilm-generating systems have revealed many of the extracellular components of biofilms, and have helped to define the various components of biofilms. Initiation of biofilm formation is characterized by the interaction of cells with a surface or interface as well as with each other. Once enough cells have aggregated, the biofilm begins to mature the final architecture of the community is guided by the extracellular matrix. Extracellular polysaccharides and proteins have been shown to be key components of the matrix. Other components, such as extracellular DNA, carbohydrate-rich polymers, and dead cells are also thought to play an important role in the establishment of biofilm structure (Branda et al., Trends in Microbiology, vol. 13, pages 20-26, 2005).

The keystone of biofilm study has been the general recognition that even single species of bacteria, when attached to surfaces and interfaces, express phenotypes that are not seen in liquid culture. For various genera whole cassettes of genes are repressed or de-repressed under the apparent control of touch receptors. Particularly, the ‘sessile phenotype’ more often displays a reduced susceptibility towards various antibacterial treatments and a more aggressive pathogenesis or corrosion potential than does the free-living ‘planktonic’ cell. Transition from the sessile to the planktonic depends on the formation of the extracellular polymeric matrix, which not only cements the bacterial cells to the surface but also maintains a spatial arrangement and helps trap many extracellular products and enzymes. The population then becomes quorate and through the mediation and accumulation of cell-cell signals (e.g., N-acyl-homoserine lactones in the case of Gram-negative bacteria) transcriptionally alters the phenotype of member cells. Cross signaling between different species and genera allows complex, multi-functional consortia to become established. For an analysis of the signaling pathways involved in biofilm formation, see Donlan et al., Emerging Infectious Diseases, vol. 8, pages 881-890, 2002. The precise nature of the colonizing species and its relationship with other bacteria determines the nature of the biofilm formed. In dental plaque such inter-relationships have been well characterized and similar processes are involved wherever surfaces are exposed to environmental microorganisms.

Methods are known within the art for preventing and/or inhibiting biofilm formation and development. For example, published U.S. patent application No. 2005/0143286 discloses a method for preventing or inhibiting biofilm formation using proteinaceaous and non-proteinaceous metal chelators. U.S. 2006/0018945 describes a method of preventing biofilm growth formation on a surface by coating or impregnating the surface with a gallium-containing composition. U.S. 2003/0134783 describes the use of cyclic heptapeptides for inhibiting biofilm formation. U.S. 2006/0030539 describes using compounds that inhibit the formation or polymerization of actin in order to inhibit biofilm formation. See also U.S. Pat. No. 6,267,979, U.S. Pat. No. 6,086,921, and U.S. Pat. No. 5,688,516.

Pseudomonas aeruginosa, a soil and water bacterium, can lead to opportunistic infections in immunocompromised or immunosuppressed patients such as burn patients and patients with cystic fibrosis. As described in published U.S. Application No. 2003/0109548, which is hereby incorporated by reference in its entirety, a study of iron transport and metabolism in Pseudomonas aeruginosa (Royt, 1988. Biochim. Biophys. Acta, 939: 493-502) led to the isolation of an iron-chelating siderophore associated with the cytoplasmic membrane of iron-rich cells, although this iron chelator was of unknown structure. U.S. Application No. 2003/0109548 discloses that this iron chelator involved two compounds 4-hydroxy-2-nonylquinoline and 4-hydroxy-2-heptylquinoline shown below:

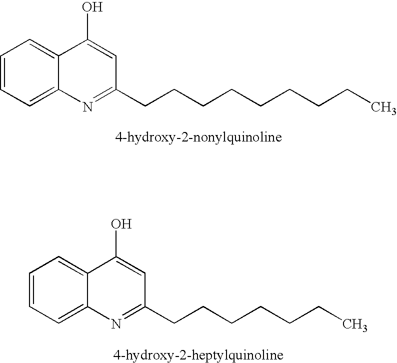

Previously isolated from the filtrates of 4 to 6 week old culture of P. aeruginosa, the 4-hydroxy-2-nonylquinoline (the C-9 compound) was originally called Pyo Ic and shown to exhibit antibiotic activity (Hays et al., J. Biol. Chem., 159: 725-749, 1945). The name pseudan was applied later (Ritter and Luckner, Eur. J. Biochem. 18: 391-400, 1971). The pseudans have been identified as having alkyl chains of C₇-C₁₂, e.g. pseudan IX (Herbert, 1989, 197. The Biosyntheses of Secondary Metabolites. 2nd ed. Chapman and Hall. New York).

U.S. 2003/0109548 further discloses that 4-hydroxy-2-alkylquinolines can be prepared by isolation from natural sources, such as P. aeruginosa, using conventional methods, e.g., electrophoresis, mass spectrometry, liquid chromatography, HPLC, thin-layer liquid chromatography, detergent extraction (e.g., non-ionic detergent, Triton X-100, CHAPS, octylglucoside, Igepal CA-630), ethanol extraction, ammonium sulfate or ethanol precipitation, acid extraction, anion or cation exchange chromatography, phosphocellulose chromatography, hydrophobic interaction chromatography, hydroxyapatite chromatography, lectin chromatography, gel electrophoresis, immuno-purification methodology, etc. A method is described in Royt et al., Biochim. Biophys. Acta., vol. 939, pages 493-502, 1988.

Additionally, U.S. 2003/0109548 discloses that 4-hydroxy-2-alkylquinolines can be be prepared synthetically. For example, procedures for the synthesis of 4-hydroxy-2-nonylquinoline are described by Wells, J Biol. Chem., vol. 196: pages 331-340, 1952; and Somanathan and Smith, J. Heterocyclic Chem., vol. 18: page 1077, 1981. Additional synthetic methods which start with a lower alkyl 4-hydroxyquinoline, such as 4-hydroxy-2-methylquinoline, may also be employed. Reaction of this compound with sodium amide in liquid ammonia solvent gives the dianion where both aromatic substituents are deprotonated. When the dianion is further reacted with a limited amount of an alkyl halide, selective alkylation is achieved at the 2 methyl carbanion. The desired 4-hydroxy-2-alkylquinolines can be prepared upon work-up with acid.

In a more recent study on iron transport and metabolism in Pseudomonas aeruginosa, the applicants found that a decrease in total cell iron of iron-rich, late logarithmic phase was due to iron efflux which was accompanied by the appearance of aggregated particles (i.e., inclusion bodies) on pellets of centrifuged cells. Inclusion bodies are highly reflective, dense aggregates that function to compartmentalize various substances that are either foreign, toxic, or in excess to the bacterial cell. Inclusion bodies of prokaryotic cells serve various functions (for a detailed review, see Shively et al., Ann. Rev. Microbiol., vol. 28: pages 167-187, 1974). Some inclusion bodies are storage sites for carbon and energy sources. For example, lipid inclusion bodies consisting of poly-β-hydroxybutyric acid or other polyhydroxyalkanoates, triacylglycerols, and wax esters provide carbon and energy to the cell. Some bacteria derive energy upon oxidizing glycogen or starch found in polysaccharide inclusion bodies or sulfur found in sulfur inclusions bodies, whereas other organisms store inorganic phosphate in inclusions to be used in the synthesis of ATP. Other inclusion bodies carry out more specialized functions such as carbon dioxide fixation during photosynthesis, buoyancy, proper orientation along a magnetic field. A characteristic of prokaryotic inclusion bodies is the absence of a unit membrane defining them. Rather, these structures may be enclosed by a non-unit lipid membrane or by protein. Some inclusion bodies are located in the cytoplasm of the cell, whereas others are found in the periplasmic space.

In general, the above-mentioned inclusion bodies involved in iron transport and metabolism in Pseudomonas aeruginosa comprise one or more pseudan species (e.g., the C7 and/or C9 forms), 3,4-dihydroxy-2-heptylquinoline (the Pseudomonas quinolone signaling compound, PQS), and one or more forms of iron (e.g., Fe⁺³ and salts thereof). For example, the inclusion bodies can contain iron, pseudan I (4-hydroxy-2-methylquinoline), pseudan VII (4-hydroxy-2-heptylquinoline), pseudan IX (4-hydroxy-2-nonylquinoline), 3,4-dihydroxy-2-heptylquinoline, and 4-hydroxy-2-nonylquinoline N-oxide. These aggregated particles were determined to be iron-containing intracellular inclusion bodies which, among other things, affect biofilm formation. Among the quinoline derivatives found in such inclusion bodies pseudan I, VI, and IX were found in relative mole-based amounts of 1.00, 0.99, and 1.10. The 1:1:1 stoichiometry of pseudan I, VII, and IX suggests the complexation ratio of chelators to iron. The size of the inclusion bodies can vary. For example, the size of the inclusion bodies in the cells range from 25 to 50 nm.

These pseudan inclusion bodies were found to result in a decrease in cell aggregation. While not being bound to any particular theory, it is believed rhamnolipids on the surface of P. aeruginosa solubilze the inclusion bodies resulting in decrease cell adherence to surfaces.

Thus, according to one aspect of the invention there is provided a method of preventing or inhibiting the growth or development of a biofilm produced by bacteria, comprising contacting the biofilm with an effective amount a pseudan inclusion body.

According to a further aspect of the invention, there is provided a method of preventing or inhibiting the growth or development of a biofilm on a surface, biotic or abiotic, comprising applying to the surface an effective amount of pseudan iron-inclusion bodies, wherein said pseudan inclusion bodies comprise iron, 3,4-dihydroxy-2-heptylquinoline, and at least one pseudan species selected from 4-hydroxy-2-alkylquinolines in which the alkyl group contains 1-12 carbon atoms, e.g., 7-12 carbon atoms.

According to a further aspect of the invention, there is provided a method of preventing or inhibiting the growth of a biofilm on a surface, biotic or abiotic, comprising contacting the surface with an effective amount a pseudan inclusion body which has iron-binding properties.

According to a further aspect of the invention, there is provided a method of preventing or inhibiting the growth of a biofilm on a surface, biotic or abiotic, comprising contacting the surface with an effective amount a pseudan inclusion body which has iron-binding properties, wherein said pseudan inclusion bodies comprise 3,4-dihydroxy-2-heptylquinoline and at least one pseudan species selected from 4-hydroxy-2-alkylquinolines in which the alkyl group contains 1-12 carbon atoms, e.g., 7-12 carbon atoms.

A further aspect of the invention is a composition of isolated pseudan inclusion bodies optionally in combination with at least one carrier, such as a pharmaceutically acceptable carrier. In addition, the composition may further comprise other antibacterial agents. By “isolated” it is meant that the inclusion bodies are essentially free of the components in which they are found with in nature.

When the surface involved is a biotic surface, it is preferable that the inclusion bodies do not include 3,4-dihydroxy-2-heptylquinoline. In particular, the pseudan inclusion bodies used to treat biotic surfaces comprise comprise Fe³⁺, 4-hydroxy-2-methylquinoline, 4-hydroxy-2-heptylquinoline, and 4-hydroxy-2-nonylquinoline.

In accordance with the invention, the surface to be treated with the pseudan inclusion bodies can be, for example, the surface of a medical device or a surface used in an industrial situation such as the pipelines in a water treatment plant or system. When the surface is that of a medical device, the inclusion bodies preferably do not include 3,4-dihydroxy-2-heptylquinoline.

Medical devices that can be treated in accordance with the inventive methods include, for example, catheters such as vascular catheters, epidural catheters, peritoneal catheters, and urinary catheters. The device can also be a shunt, a stent (such as a biliary stent, vascular stent or urinary stent), an orthopedic device, a prosthetic device, pacemaker, heart valve, pump, implant, endotracheal device, guidewire, optical or ocular lens, dental device, or surgical device. The device can also be one used for the storage or transportation of biological or medical fluids such as as an inhaler, syringe, or ampule.

According to a further aspect of the invention, there is provided a method of treating and/or prevent bacterial infections, comprising contacting a surface, biotic or abiotic, or administering to a patient an anti-bacterial pseudan composition comprising pseudan I (4-hydroxy-2-methylquinoline), pseudan VII (4-hydroxy-2-heptylquinoline), pseudan IX (4-hydroxy-2-nonylquinoline), and optionally Fe⁺³. Preferably, the pseudan composition contains pseudan I, VI, and IX at a stoichiometric ratio of 1:1:1.

According to a further aspect of the invention, there is provided an anti-bacterial pseudan composition comprising pseudan I (4-hydroxy-2-methylquinoline), pseudan VII (4-hydroxy-2-heptylquinoline), pseudan IX (4-hydroxy-2-nonylquinoline), and optionally Fe⁺³. Preferably, the pseudan composition contains pseudan I, VI, and IX at a stoichiometric ratio of 1:1:1.

Inclusion bodies useful in the present invention can be collected either from the extracellular milieu or by lysing the cells. In the case of extracellular inclusion bodies routine techniques may be applied in the isolation and purification. For e.g., centrifugation of a 24-hour bacterial culture at 2,987×g for 10 min at 4° C. to initially pellet the bacteria followed by filtering the spent media through a 0.45 μm filter. Ultra-centrifugation of the filtrate at 265,000×g (Ti70 rotor, Beckman Coulter Instruments) for 2 h at 4° C. pellets the inclusion bodies. The resultant pellet can then be washed with MOPS buffer and solubilized to isolate its contents or alternatively, stored at −80° C. until further experimentation. For purification of the contents, the isolated inclusion bodies are treated with acidified ethyl acetate following the procedure used by others (McGrath et al., FEMS Microbiol. Letrs., vol. 230: pages 27-34, 2004) to isolate PQS from spent media, and/or treating the cultures with P. aeruginosa rhamnolipid, which is commercially available from Jeneil Biosurfactant Co., Saukville, Wis.. Solubilization of the inclusion bodies is facilitated by vortexing for 30 min. Various kits and methods may also be employed, either solely or in combination with the above-said method for the extraction of intracellular inclusion bodies. For e.g., Seely et al. (U.S. Pat. No. 4,656,255) describes a method for extracting inclusion bodies of E. coli for the purification of protein aggregates.

Yet another possible method may rely on the isolation of minute vesicles (about 50 nm in diameter) released from the outer membrane by Gram-negative bacteria (Mashburn et al., Nature vol. 437: pages 422-425, 2005). The vesicles consist of a lipid bilayer surrounding an aqueous core and can also transport PQS. In P. aeruginosa, PQS is one of at least 55 quinolones and quinolines synthesized by the bacteria, some of which have potent antibacterial activity and signaling properties. The production of these vesicles is regulated by PQS. Fractionation and purification of such vesicles allows for a simple method for the isolation of the contents of the inclusion bodies.

According to a further aspect of the invention, there is provided a method of preventing the growth of a biofilm on a surface, biotic or abiotic, comprising contacting the surface with an effective amount of pseudan inclusion bodies which have iron-binding properties. Any biofilm produced by bacteria can be prevented or reduced and/or inhibited. Although not wishing to be bound by any particular theory, it is believed that the bacterium to be treated preferably are capable of solubilizing the pseudan inclusion bodies. Preferably, the methods are used to inhibit/prevent the growth of biofilms produced by Pseudomonas, such as Pseudomonas aeruginosa, although the methods can be used against other biofilm-forming bacteria, including E. coli, Staphylococcus, and other gram-positive and gram-negative. Any biofilm can be inhibited in accordance with the present invention, including biofilms that form on artificial surfaces and natural surfaces, such as in lungs (e.g., in cystic fibrosis (CF) patients), burns or other wounds, etc.

The present invention also relates to the use of pseudan-inclusion bodies to treat and/or prevent bacterial infections, comprising: contacting a bacterium with a composition comprising pseudan-inclusion bodies. Any bacteria can be treated, including E.coli, Staphylococcus, Pseudomonas, and other gram-positive and gram-negative. The bacteria can be planktonic, sessile, in a biofilm, in a wound, on a substrate (e.g., a stent or wound drain), etc. The composition can be administered as a liquid, cream, powder, impregnated in a wound dressing, or in any form that is suitable for treating and/or preventing infection.

Again, while not wishing to be bound by any particular theory, in inhibiting the growth of the biofilm it is quite possible that the pseudan inclusion bodies are promoting release of cells from each other and/or from a surface. While this, of course, reduces the biofilm at that location, it also leads to the production of planktonic cells that could to go downstream to colonize at a different location. On the other hand, the planktonic cells, which are outside the polysaccharide matrix of the biofilm, are more susceptible to drug treatments such as treatments with antibiotics and/or antibacterial agents.

When used to treat a biofilm in a patient or prevent the formation of a biofilm in a patient, it would be desirable to remove PQS, 3,4-dihydroxy-2-heptylquinoline, from the pseudan inclusion bodies as the presences of PQS could promote disease caused by P. aeruginosa. One method to do this would be to synthetically prepare the pseudan inclusion bodies using conventional techniques from the pseudans and Fe⁺³. For example, pseudan I, VI, and IX can be combined with Fe³⁺ within a lipid vessel (e.g., a liposome) at a stoichiometry ratio of, for example, 1:1:1.

Biofilms assays can be performed routinely, e.g., by culturing Pseudomonas on polystyrene or other appropriate substrate, and then applying crystal violet to stain and/or quantitate the biofilm. Quantitative biofilm production assays may comprise measuring bacterial cell turbidity using a microtiter plate reader (Bio-Rad, Richmond, Calif.). The assay is carried out at an optical density at 595 nm (OD₅₉₅) at different time intervals. With P. aeruginosa, biofilms form within minutes. Thus, suitable time intervals for P. aeruginosa biofilms are within 60-90 min. Alternatively, a fluorescence-based or an enzymatic quantitative assay may be employed for measuring the production of biofilms and/or as a means of screening for clinical isolates (Butterfield et al., J. Microbiol Methods, vol. 50, pages 23-32, 2002).

The inclusion bodies can be applied to a surface either alone or as a composition comprising the inclusion bodies and an acceptable carrier. For example, the composition can be applied to the surface by coating or spraying, or the surface can be immersed in a liquid composition. Procedures and techniques for treating surfaces (both biotic and abiotic) with compositions to inhibit and/or prevent biofilms are described in, for example, the published US applications discussed above, such as 2005/0143286 and U.S. 2006/0018945.

When used to treat a patient suffering from a bacterial infection or when used to treat a biofilm in a patient (e.g., a human) (such as a biofilm in the lungs of a human, e.g., a cystic fibrosis patient), the inclusion bodies or pseudan composition (e.g., a combination of pseudan I, pseudan VII, pseudan IX, and optionally Fe⁺³) can be used in the form of a composition comprising the inclusion bodies or the pesudans and at least one pharmaceutically acceptable carrier.

The carrier must be “acceptable” in the sense of being compatible with the other ingredients of the formulation and not being deleterious to the surface to which they are applied or to the recipient to which they are administered. The composition can further contain conventional excipients used in pharmaceutical formulations. The pharmaceutical acceptable carrier may also provide a controlled release formulation that is capable of slowly releasing a composition of the present invention into a patient. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems.

Pharmaceutical compositions include those suitable for oral, rectal, nasal, topical, transdermal, vaginal or parenteral (including intramuscular, sub-cutaneous and intravenous) administration or in a form suitable for administration by inhalation or insufflation (such as an aerosol). Pharmaceutically acceptable carriers are well known in the art and include those materials or vehicles which are not harmful to the individual and are suitable for administering compounds of the present invention to patient (e.g., mammals). Carriers include liquid or solid filler, diluent, excipient, solvent or encapsulating material. Examples of suitable pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

When used as a pharmaceutical, the amount of the inclusion bodies or pseudan combination present in the administered composition will vary based on the route of administration, the nature of the condition for which treatment is required, and the age and condition of the patient and will be ultimately at the discretion of the attendant physician or veterinarian. The amount of inclusion bodies to be administered to a patient or applied to a surface can be easily determined by one of skill in the art using routine experimentation.

Pharmaceutical compositions suitable for oral administration may conveniently be presented as discrete units such as capsules, cachets or tablets each containing a predetermined amount of the active ingredient; as a powder or granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The inclusion bodies or pseudan combination may also be formulated for parenteral administration (e.g., by injection, for example, bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain additional formulation agents such as suspending, stabilizing and/or dispersing agents. Alternatively, the inclusion bodies can be in powder form, obtained by aseptic isolation of sterile solid or by lyophilisation from solution, for constitution with a suitable vehicle, e.g., sterile, pyrogen-free water, before use.

For topical administration to the epidermis, the psuedan inclusion bodies or pseudan combination may be formulated as ointments, creams or lotions, or as a transdermal patch. Such transdermal patches may contain penetration enhancers such as linalool, carvacrol, thymol, citral, menthol and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

The psuedan inclusion bodies or pseudan combination can be administered as an aerosol formulation. Suitable for aerosol delivery include powders, microcapsules, microparticles, liposomes, and nebulized sprays. Aerosol formulations can be administered by, for example, nebulizers and inhalers such as pressurized metered dose inhalers (MDI), dry powder inhalers (DPI), and metered solution devices (MSI).

The entire disclosures of all applications, patents and publications, cited above and below, are hereby incorporated by reference.

### BRIEF DESCRIPTION OF THE DRAWINGS

FIG. 1. Cell growth, total cell iron, and extractable pseudan in three substrains of P. aeruginosa: A) 15692a; B) 15692b; C) 15692d. At each time period, the optical density was determined, and two aliquots of 25×109 cells were collected to determine concentrations of total cell iron and extractable pseudan.

FIG. 2. Efflux of ⁵⁵FeCl3 from substrain 15692d. A) cell density: B) total cell ⁵⁵Fe: C) ⁵⁵Fe in 200 μl of spent media before transfer of cells and in the filtrate after transfer of cells.

FIG. 3. A high resolution electrospray mass spectrum of whole inclusion bodies.

FIG. 4. Electron impact mass spectrum of the 23.54 minute peak of the GC-MS of whole inclusion bodies. The sample was dissolved in methanol.

FIG. 5. A) SEM back-scattered electron image of 24 hr P. aeruginosa 15692d cells. Bar=1 μm. B) Elemental analysis of whole cells. C) Elemental analysis of the bright spot.

FIG. 6. Cell growth, total cell iron, and iron in inclusion bodies in A)15692a and B) 15692d cells. Total cell iron in 25×109 cells and in washed inclusion bodies was measured after acid hydrolysis.

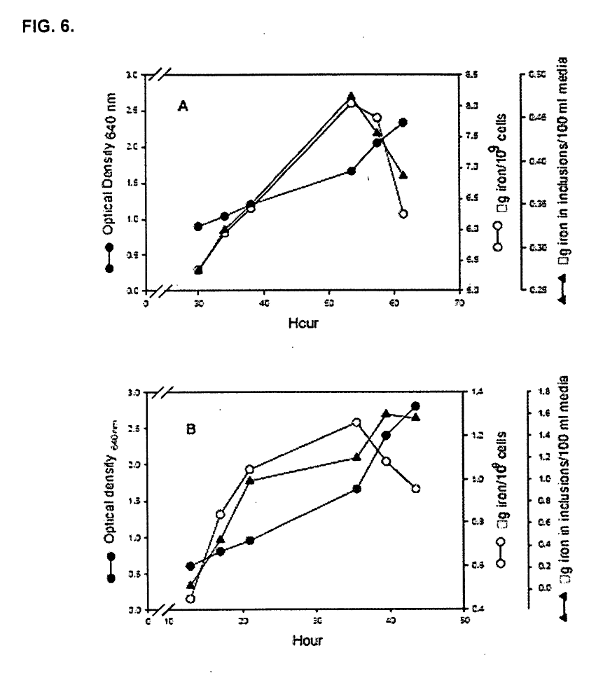

FIG. 7. Adherence of cells to polystyrene microtiter plate wells and levels of extractable pseudan. The optical density at 600 nm is the average of solubilized crystal violet from three wells after 60 min incubation. Bars represent the range. A) 15692a cells. B) 15692b cells. C) 15692d cells.

FIG. 8. Adherence of A) 5 hr, B) 25 hr, and C) 30 hr 15692b cells to polystyrene microtiter plate wells in the absence (closed bars) and presence of inclusion bodies containing 31 μg iron (light grey bars) and 62 μg (dark-grey bars) iron. The optical density at 600 nm is the average of solubilized crystal violet from three wells after 60 min incubation. Bars represent the range.

### EXAMPLES

In the foregoing and in the following examples, all temperatures are set forth uncorrected in degrees Celsius; and, unless otherwise indicated, all parts and percentages are by weight.

Bacteria and Culture Methods.

Three substrains of Pseudomonas aeruginosa ATCC 15692 were used in all experiments. These substrains are designated 15692a, 15692b, and 15692d. Cultures were maintained at −70° C. in Luria-Bertani broth containing 25% glycerol. Cells were grown in trypticase soy broth (TSB: DIFCO Laboratories), shaking at 100 rpm at 30° C. in a New Brunswick rotatory shaker. Typically, cultures were inoculated with logarithmic phase cells to an initial optical density of 0.02 to 0.04. Cells were harvested by centrifugation at 11,950×g for 10 min at 4° C., and washed two times with 60 mM 3-(4-Morpholino) Propane Sulfonic Acid buffer (MOPS buffer), pH 7.2.

Iron and Pseudan Determination.

25×109 cells were harvested in acid-washed centrifugetubes and washed two times as stated above. To determine total iron concentration, a pellet ofcells was acid hydrolyzed with nitric acid/ perchloric acid (1/1: v/v). Following dilution in 0.1 MTris-HCI buffer, pH 7.8, iron was determined on a Perkin-Elmer 5001 PC Atomic Absorption Spectrophotometer. Total iron concentration consists of intracellular iron, iron in periplasmic inclusion bodies, and iron in aggregates of inclusion bodies pelleted with cells. The iron inisolated inclusion bodies was also determined after acid hydrolysis as above.

Pseudan VII and IX were extracted from a pellet of cells with ethanol as before (Royt et al., Biochim. Biophys. Acta, vol. 939: pages 493-502, 1988). Here, 25×109 cells were extracted with 1 ml ethanol for 1 h, followed by centrifuging at 38,720×g for 30 min at 4° C. This extraction procedure was repeated two more times, and the extracts combined. The concentration of pseudan VII and IX was determined spectrophotometrically upon measuring the spectrum of synthesized pseudan IX with a millimolar extinction coefficient of 151.5 mM⁻¹ cm⁻¹ at 231 nm. In this study, extractable pseudan consists of pseudan VII and IX in the pellet of cells, that is the membrane-bound compounds, that which is extractable from periplasmic inclusion bodies, and that which is extractable from extracellular, aggregated inclusion bodies. Efflux experiment. Two flasks each containing 50 ml TSB were inoculated with logarithmicphase cells as described above. Fifty μCi ⁵⁵FeCl3 (New England Nuclear Corporation, Boston. Mass.) was added to one of the flasks. After shaking at 30° C. for 24 hours, each culture was centrifuged, and the radiolabeled cells were washed one time with room-temperature 60 mM MOPS buffer. The radiolabeled cells were transferred to the spent media of the unlabeledculture. At designated time intervals, 1×109 cells were transferred to a filter, washed two timeswith MOPS buffer, and the radioactivity counted. Also, 200 μl of the cell-free filtrate, i.e., the spent media, was counted at each time interval.

Collection of Extracellular Inclusion Bodies

A 24 hour culture of 15692d cells was centrifuged at 2,987×g for 10 min at 4° C. The spent media was filtered through a 0.45 μm filter, and the filtrate centrifuged at 265,000×g (Ti70 rotor, Beckman Coulter Instruments) for 2 h at 4° C. The resultant pellet was washed one time with MOPS buffer. Fractionation of the inclusion bodies by thin layer chromatography (TLC). Isolated inclusion bodies were treated with acidified ethyl acetate, following the procedure used by othersto isolate the PQS from spent media of P. aeruginosa (McGrath et al., FEMS Microbiol. Letrs. Vol. 230: pages 27-34, 2004), and with P. aeruginosa rhamnolipids obtained from Jeneil Biosurfactant Co., Saukville, Wis. Solubilization of the inclusion bodiesoccurred upon vortexing for 30 min. Samples were spotted on silica gel TLC plates that hadbeen soaked in 5% w/v KH2PO4 and activated for 1 hr at 100° C. Separation of compounds occurred using methylene chloride:acetonitrile:dioxane (17:2:1) (Pesci et al., Proc. Natl. Acad. Sci. U.S.A. vol. 96: pages 11229-11234,1999).

Electron Microscopy.

(i) Transmission electron microscopy (TEM) was performed on pellets of 5 h and 24 h 15692d cells, and on isolated inclusion bodies. Samples were resuspended in 4% formaldehyde/1% glutaraldehyde overnight at 4° C., post-fixed in 1% osmium tetroxide, dehydrated in graded alcohols and embedded in Epon 812. Thin (80-100 nm) sections werestained with lead citrate and uranyl acetate and examined in a LEO912 electron microscope (Carl Zeiss SMT, Thornwood, N.Y.).

(ii) Scanning electron microscopy (SEM) was done on 24 hr 15692d cells using a JEOL JSM-840 electron microscope with a LaB6 electron emitter. The SEM was equipped with an energy dispersive x-ray analyzer (EDAX) using a Si(Li) crystal (Princeton Gamma-Tech, Princeton, N.J.). The sample was fixed in 1% glutaraldehyde for 2 hr, and dehydrated with acetone.

Attachment Assay.

An assay for the initiation of biofilm formation was used to determineattachment of cells to wells of a polystyrene microtiter plate (O'Toole et al., Mol. Microbiol., vol. 28: pages 449-46, 1999). Cells (5×107) in spent media were added to wells of a plate in triplicate The plates were incubated at 30° C. shaking at 35 rpm. At 15 min intervals for 1 h, media and planktonic cells were removed, and the wells washed 3 times with room-temperature MOPS buffer. 250 μl 0.1% crystal violet was added to the wells for 15 min. The wells were then washed 3 times with MOPS buffer, and the dye in cellsadhered to the well surface was solubilized in 95% ethanol. The absorbance at 600 nm was thenmeasured. Controls consisting of spent media treated as above were subtracted from eachsample. When mentioned, inclusion bodies resuspended in MOPS buffer at the given concentrations were added to cells in spent media, and the attachment assay carried out. Controls of inclusion bodies resuspended in MOPS and added to spent media were subtracted from these values.

High Resolution Electrospray Ionization Mass Spectrometry (ESI MS).

Electrospray ionization mass spectra were obtained on a Waters LCT Premier Time-Of-Flight (TOF) MassSpectrometer. The instrument was operated in positive ion mode at a resolution of 10000. The electrospray capillary voltage was 3 kV and the sample cone voltage was 60 volts. The desolvation temperature was 225° C. and the desolvation gas was nitrogen at 300 L/hr. Accurate masses were obtained using the lock spray mode with Leu-Enkephalin as the external reference compound. The ESI mass spectra were obtained via the direct loop injection method.

Results

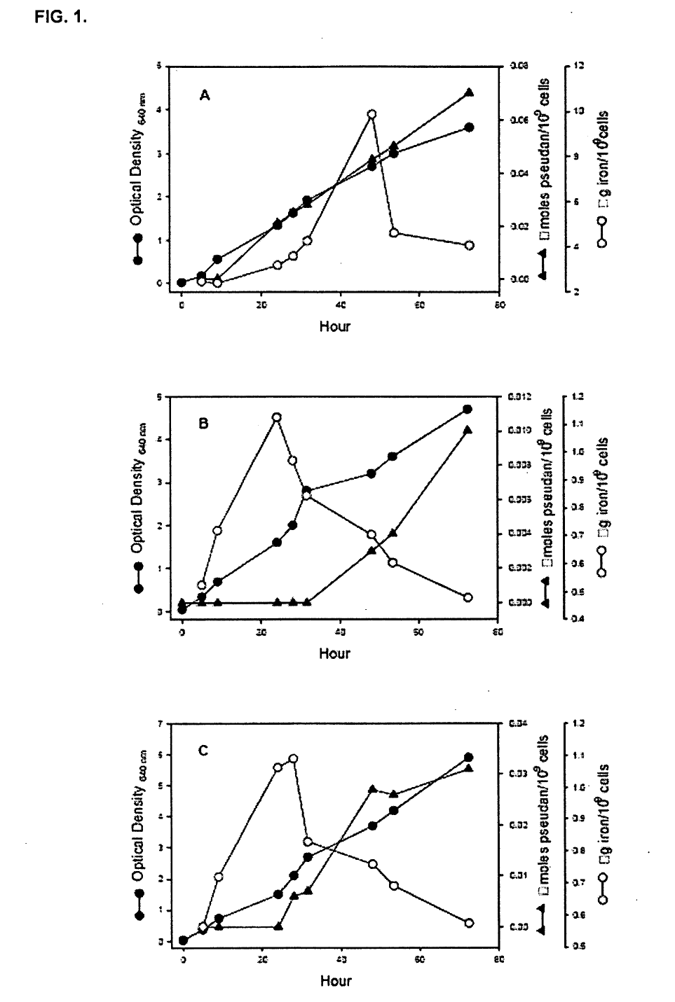

Total cell iron decreases in late logarithmic phase. Upon tracking iron levels in the three substrains of P. aeruginosa growing in iron-rich TSB, it was found that total cell iron increased in the early logarithmic phase of growth (FIG. 1). This increase in total cell iron was consistently followed by a decrease as the cell population continued into later logarithmic phase. It is noted in FIG. 1 that as total cellular iron decreased, extractable pseudan increased. Substrain 15692a had the greatest quantity of both iron and extractable pseudan, as compared to those quantities produced by substrains 15692b and 15692d. Pseudan was extractable from substrains 15692a and 15692d after approximately 24 hr, whereas pseudan was not extractable from substrain 15692b for approximately 48 h after culture inoculation, and it is noted that significantly less pseudan was extractable from substrain 15692b than from the other two substrains. The decrease in total cell iron seen with each substrain may be explained by iron efflux from the cell and/or the stoppage of iron uptake as the population of cells increased in number.

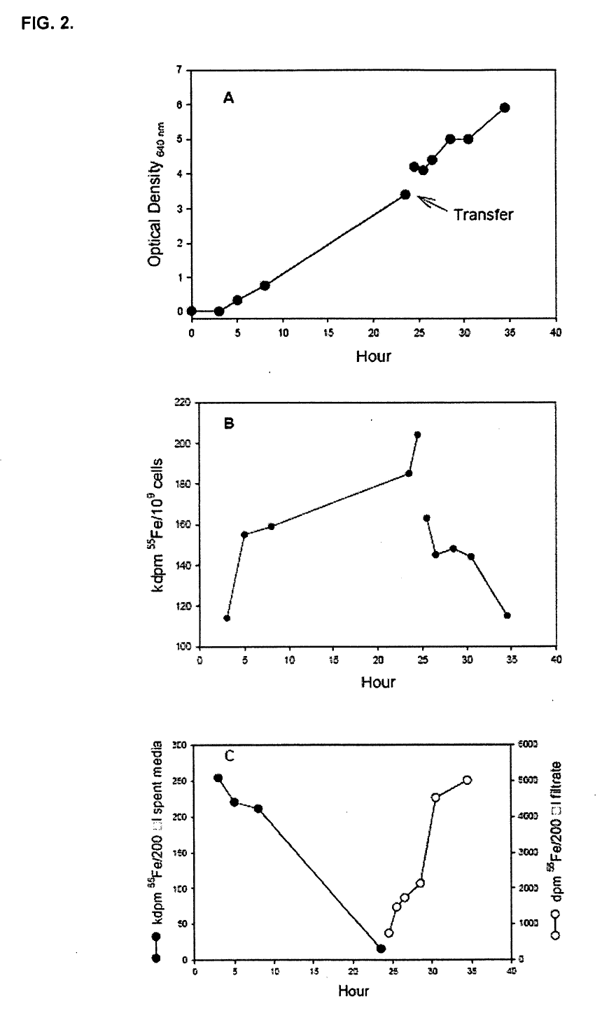

To determine if iron efflux occurs from the iron-rich cells, substrain 15692d was radiolabeled with ⁵⁵FeCl3, and transferred to ⁵⁵Fe-free spent media as described above. At set time periods, cell-associated radioactivity and radioactivity in the spent media were determined (FIG. 2). It is seen that radiolabeled iron accumulated in the cells as cell density increased. Upon transfer of radiolabeled cells to ⁵⁵Fe-free spent media, ⁵⁵Fe is released from the cells and is recovered in the media. When initially doing such efflux experiments it was noted that cells aggregated when transferred instead to fresh TSB or to MOPS buffer, resulting in erratic results of total cell iron. Such aggregation suggested that a substance in the spent media served to prevent cell aggregation.

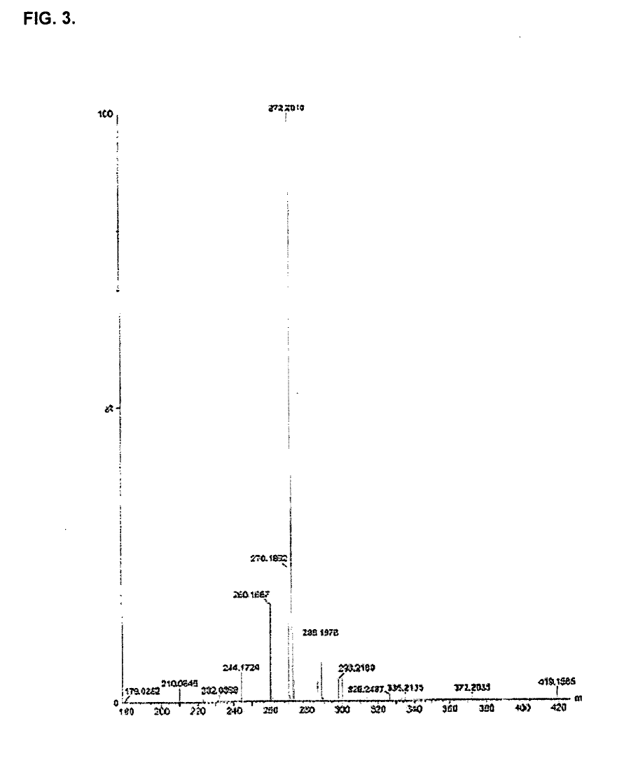

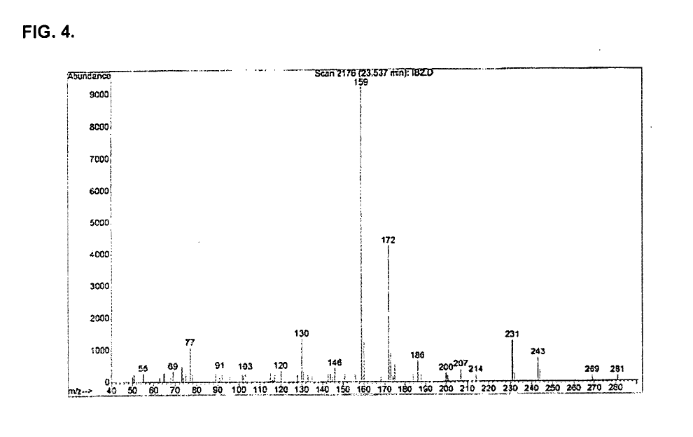

Extracellular aggregates were extracted with rhamnolipid and with acidified ethyl acetate. Thin layer chromatograms of these extracts revealed the appearance of at least four bands, one of which, Rf=0.55, was fluorescent blue. ESI MS of whole inclusion bodies (FIG. 3) showed a peak of m/z 272.2010 consistent with the C18H26NO formula, or pseudan IX. A peak was also observed at m/z 288.1978 (C18H26NO2) which may be 4-hydroxy-2-nonylquinoline-N-oxide, with establishment of structure via ease of loss of the oxygen atom on the nitrogen (Taylor et al., J. Chromatogr. vol. 664: pages 458-462, 1995). The peak of m/z 270.1862 (C18H24NO formula) is of dehydrated, 4-hydroxy-2-nonylquinoline-N-oxide, and a peak at m/z 244.1724 (C16H22NO) is pseudan VII, and finally, a peak at 260.1667 (C16H22NO2 formula) is 3,4-dihydroxy-2-heptylquinoline. Absent from the spectra is dehydrated, 4-hydroxy-2-heptylquinoline-N-oxide which would have a molecular weight of 242. This absence suggests that the position of the second oxygen atom in the two compounds, i.e., the C7 compound and the C9 compound differs, in that one is a protonated N-oxide, as in the C9 compound, and the other a hydroxyl group. Hence, the peak at 260.1667 is that of 3,4-dihydroxy-2-heptylquinoline. (iii) GC-MS was also carried out using the whole inclusion bodies, and using the blue fluorescent spot eluted from the thin layer chromatogram. The total ion chromatogram (TIC) of the whole inclusion bodies revealed about 20 major peaks. Pseudan VII was identified at 23.54 minutes, and pseudan IX was identified at 23.90, 24.02, and 25.20 minutes. 4-Hydroxy-2-nonylquinoline-N-oxide and dehydrated 4-hydroxy-2-nonylquinoline-N-oxide were also evident. The TIC did not reveal the presence of 3,4-dihydroxy-2-heptylquinoline, but did show the presence of dehydrated 3,4-dihydroxy-2-heptylquinoline. Also present in the TIC at 23.54 minutes as well as in other peaks is 4-hydroxy-2-methylquinoline, or pseudan I. FIG. 4 is the mass spectrum of the 23.54 minute peak showing pseudan I, m/z 159. The TIC of the whole inclusion bodies also revealed m/z 56 in several peaks, which may be iron or an organic fragment.

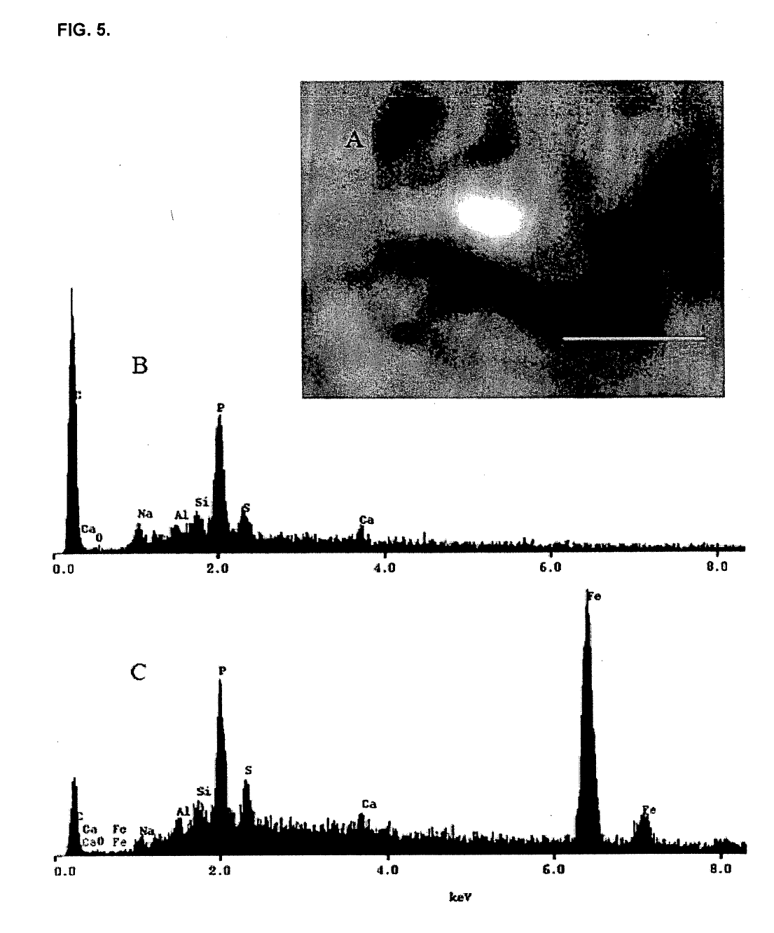

To verify that the inclusion bodies seen inside the cells are indeed iron-containing and hence are the iron containing pelleted aggregates isolated from the cells, an elemental analysis of intracellular inclusion bodies was compared with that of cells lacking inclusion bodies. FIG. 5A shows the back-scattered electron image of 24 hour old 15692d cells, one of which contains a bright area. The elemental analysis of a whole cell itself shows large amounts of carbon and phosphate, and modest amounts of other elements (FIG. 5B). The elemental analysis of the bright area reveals a high concentration of iron in addition to the elements found in the cell itself (FIG. 5C). The two iron peaks identify the types of x-rays produced: the higher energy peak is a K-beta peak in that it represents the x-rays emitted by M-shell electrons going to the K-shell, and the lower energy peak is a K-alpha peak representing x-rays emitted from electrons going from the L-shell to the K-shell.

To determine whether the iron efflux seen in FIG. 1 was mediated by the inclusion bodies, each substrain was harvested during a 48 h period including the time of iron efflux seen in FIG. 1. At each time period, total iron concentration in the pellet of cells was measured, as was the iron in inclusion bodies isolated from the spent media. It is seen in both 15692a and 15692d cells that as total cell iron increased, the iron of inclusion bodies collected from the spent media also increased. As total cell iron decreased in the 15692a cells, inclusion body iron also decreased. However, this decrease in inclusion body iron with a decrease in total cell iron did not occur in the 15692d cells. In similar studies using the 15692b cells, the iron in inclusion bodies was immeasurable using the conditions described, indicating that this substrain failed to release significant levels of inclusion bodies or that released inclusion bodies were solubilized upon being released.

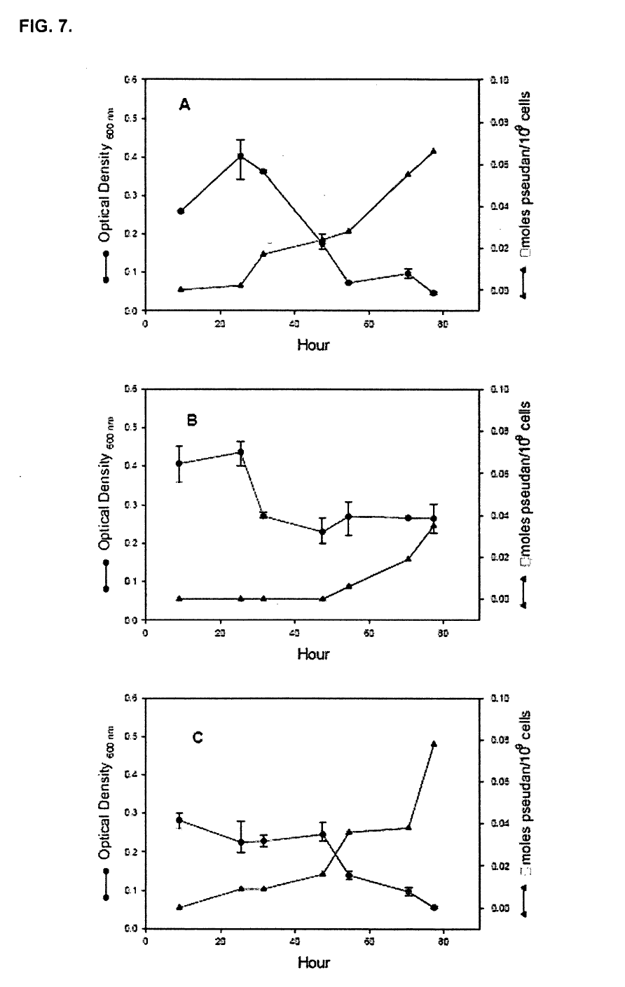

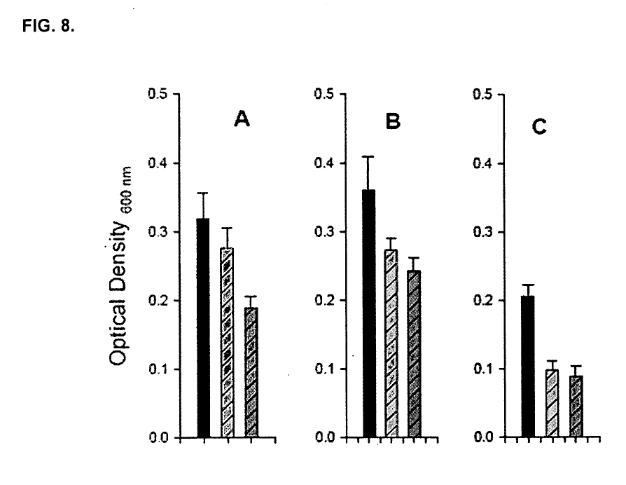

When doing efflux experiments, it was noted that transfer of washed cells to buffer or to fresh media resulted in cell aggregation. Such aggregation was minimized, however, when washed cells were transferred to spent media that contained inclusion bodies. Therefore, adhesion assays were performed to determine if the inclusion bodies played a role in preventing cell aggregation. FIG. 7 shows that with each substrain, an inverse relationship exists between cell adherence and extractable levels of pseudan, in that as the level of extractable pseudan increased with time of growth, cell adherence decreased. Also it is seen that the 15692b cells that produce the least amount of extractable pseudan show the greatest amount of cell adherence. To determine if the inclusion bodies were playing a role in preventing cell adherence, logarithmic phase 15692b cells, cells from which pseudan is not extractable and which are not producing collectible inclusion bodies, were incubated with inclusion bodies. It is seen in FIG. 8 that addition of inclusion bodies to these cells resulted in a decrease in attachment of the cells to the wells.

Attached hereto as an Appendix is a copy of unpublished manuscript describing in further detail the study on iron transport and metabolism in Pseudomonas aeruginosa. The Appendix is hereby incorporated by reference in its entirety.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

We claim:

1. A method of inhibiting the growth of a biofilm on a surface comprising applying to the surface an effective amount of pseudan iron-inclusion bodies, wherein said pseudan inclusion bodies comprise iron, 3,4-dihydroxy-2-heptylquinoline, and at least one pseudan species selected from 4-hydroxy-2-alkylquinolines in which the alkyl group contains 1-12 carbon atoms.

2. A method according to claim 1, wherein said at least one pseudan species is selected from 4-hydroxy-2-alkylquinolines in which the alkyl group contains 7-12 carbon atoms.

3. A method according to claim 1, wherein said at least one pseudan species is from 4-hydroxy-2-heptylquinoline.

4. A method according to claim 1, wherein said at least one pseudan species is from 4-hydroxy-2-nonyllquinoline.

5. A method according to claim 3, wherein said pseudan iron-inclusion bodies contain 4-hydroxy-2-nonyllquinoline.

6. A method according to claim 1, wherein said pseudan iron-inclusion bodies contain Fe³⁺, 4-hydroxy-2-methylquinoline, 4-hydroxy-2-heptylquinoline, 4-hydroxy-2-nonylquinoline, 3,4-dihydroxy-2-heptylquinoline, and 4-hydroxy-2-nonylquinoline N-oxide.

7. A method according to claim 1, wherein said biofilm is a biofilm produced by bacteria of the Pseudomonas genus.

8. A method according to claim 1, wherein said biofilm is a biofilm produced by Pseudomonas aeruginosa.

9. A method of inhibiting the growth of a biofilm on a surface comprising applying to the surface an effective amount of pseudan iron-inclusion bodies, wherein said pseudan inclusion bodies comprise Fe³⁺, 4-hydroxy-2-methylquinoline, 4-hydroxy-2-heptylquinoline, 4-hydroxy-2-nonylquinoline.

10. A method according to claim 9, wherein said surface is a biotic surface.

11. A method according to claim 9, wherein said pseudan inclusion bodies further comprise 4-hydroxy-2-nonylquinoline N-oxide.

12. A method according to claim 10, said biofilm is in the lungs of a cystic fibrosis patients.

13. A method of preventing the growth of a biofilm on a surface, biotic or abiotic, comprising contacting the surface with an effective amount pseudan inclusion bodies wherein said pseudan inclusion bodies comprise iron, 3,4-dihydroxy-2-heptylquinoline, and at least one pseudan species selected from 4-hydroxy-2-alkylquinoline in which the alkyl group contains 1-12 carbon atoms.

14. A method according to claim 13, wherein said at least one pseudan species is selected from 4-hydroxy-2-alkylquinolines in which the alkyl group contains 7-12 carbon atoms.

15. A method of preventing the growth of a biofilm on a surface comprising applying to the surface an effective amount of pseudan iron-inclusion bodies, wherein said pseudan inclusion bodies comprise Fe³⁺, 4-hydroxy-2-methylquinoline, 4-hydroxy-2-heptylquinoline, and 4-hydroxy-2-nonylquinoline.

16. A composition comprising isolated pseudan inclusion bodies optionally in combination with at least one pharmaceutically acceptable carrier, wherein said pseudan inclusion bodies comprise Fe³⁺, 4-hydroxy-2-methylquinoline, 4-hydroxy-2-heptylquinoline, and 4-hydroxy-2-nonylquinoline.

17. A composition according to claim 16, wherein said pseudan inclusion bodies further comprise 4-hydroxy-2-nonylquinoline N-oxide.

18. A composition according to claim 16, further comprising at least one additional antibacterial agent.

19. An anti-bacterial pseudan composition comprising pseudan I (4-hydroxy-2-methylquinoline), pseudan VII (4-hydroxy-2-heptylquinoline), pseudan IX (4-hydroxy-2-nonylquinoline), and optionally Fe⁺³.

20. A coated or impregnated medical device comprising a medical device in which at least one surface thereof is coated or impregnated with pseudan inclusion bodies, wherein said pseudan inclusion bodies comprise Fe³⁺, 4-hydroxy-2-methylquinoline, 4-hydroxy-2-heptylquinoline, and 4-hydroxy-2-nonylquinoline.

21. A coated or impregnated medical device comprising a medical device in which at least one surface thereof is coated or impregnated with an anti-bacterial pseudan composition comprising pseudan I (4-hydroxy-2-methylquinoline), pseudan VII (4-hydroxy-2-heptylquinoline), pseudan IX (4-hydroxy-2-nonylquinoline), and optionally Fe⁺³.
